(19) United States
(12) Patent Application Publication
(10) Pub. No.: US 2022/0017290 A1
(43) Pub. Date: Jan. 20, 2022

(54) Face Mask Dispenser

(71) Applicant: Christine Carole Groethe, Edgewater, CO (US)

(72) Inventor: Christine Carole Groethe, Edgewater, CO (US)

(21) Appl. No.: 17/375,435

(22) Filed: Jul. 14, 2021

Related U.S. Application Data
(60) Provisional application No. 63/053,981, filed on Jul. 20, 2020.

(51) Int. Cl. B65D 83/08

(52) U.S. Cl. CPC B65D 83/0817; B65D 83/0894

(57) ABSTRACT

The facemask dispensing device an apparatus that is intended to dispense a protective equipment such as masks to a user before entering a building or other location. The dispenser allows a user to extract a single use mask without having to touch other surfaces or masks contained in the dispenser. The dispenser is provided a body which houses a plurality of masks and a mounting plate that may allow for the dispenser to be mounted onto a wall. The plurality of masks may be push towards a front of the dispenser to an opening where a user can simply grab a mask to wear. Each of the plurality of masks may be contained in a package to further prevent contamination of other masks positioned inside the dispenser. Furthermore, the dispenser may be positioned in a vertical manner instead of being mounted onto a wall.

The current application claims a priority to the U.S. Provisional Patent application Ser. No. 63/053,981 filed on Jul. 20, 2020.

### FIELD OF THE INVENTION

The present invention relates generally to a type of dispenser. More specifically, the present invention is a dispenser that may dispense face masks or other personal protective equipment.

### BACKGROUND OF THE INVENTION

With an onslaught of more deadly and infectious diseases, it may be beneficial to introduce more protocols and standards to help hinder the spread of such disease. Wearing protective gear such as masks, face shields, and gloves have been proven to help prevent the spread of viruses by physically hindering the distance and path in which these particles may be able to reach mucous membranes such as those of the eyes, nose, and mouth. However, it can be difficult to offer those without protective gear, the right protection to gain access to particular buildings or locations.

An objective of the present invention is to create an apparatus which may dispense protective equipment such as masks to a user before entering a building or other location. The dispenser allows a user to extract a single use mask without having to touch other surfaces or masks contained in the dispenser. The dispenser is provided a body which houses a plurality of masks and a mounting plate that may allow for the dispenser to be mounted onto a wall. The plurality of masks may be pushes towards a front of the dispenser to an opening where a user can simply grab a mask to wear. Each of the plurality of masks may be contained in a package to further prevent contamination of other masks positioned inside the dispenser. Another method of dispensing masks from the present invention is by having a hook and loop fastener tab that attaches to a mask contained in a package. The user may simply pull a mask out which detaches the mask from the container. A dish may be positioned underneath an opening of the dispenser to catch any masks that could potentially fall. This also prevents the mask from falling out of the dispenser. Furthermore, the dispenser may be positioned in a vertical manner instead of being mounted onto a wall.

### BRIEF DESCRIPTION OF THE DRAWINGS

FIG. 1 is a top-front perspective view of the present invention, wherein the lid has a slit.

FIG. 2 is a top-front perspective view of the present invention showing a plurality of masks within the storage compartment.

FIG. 3 is a front elevational view of the present invention.

FIG. 4 is a sectional view of the present invention, taken along A-A′ of FIG. 3.

FIG. 5 is a top front perspective view of the present invention, wherein a catch plate is positioned adjacent the storage compartment.

FIG. 6 is a bottom perspective view of the present invention, wherein the lid is in an open configuration.

### DETAIL DESCRIPTIONS OF THE INVENTION

All illustrations of the drawings are for the purpose of describing selected versions of the present invention and are not intended to limit the scope of the present invention.

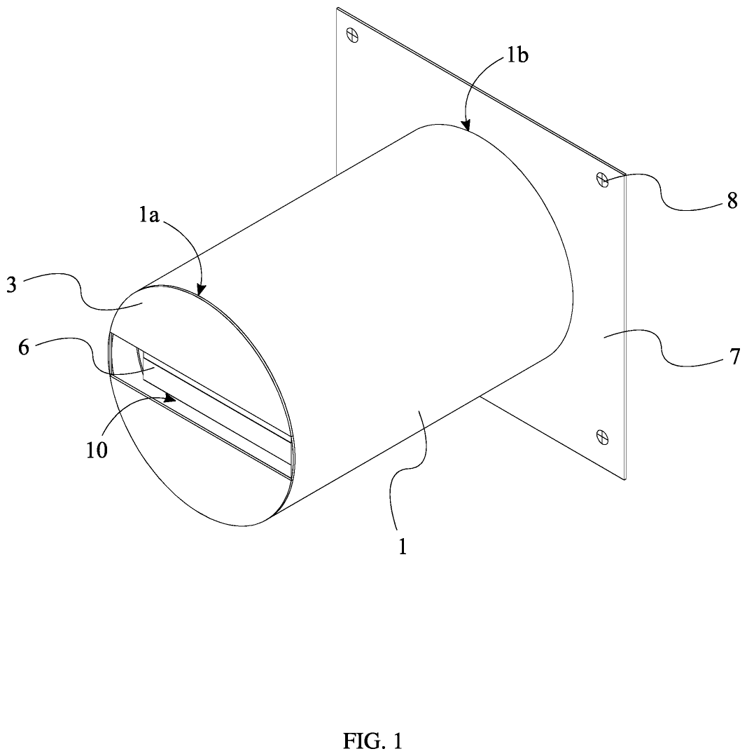

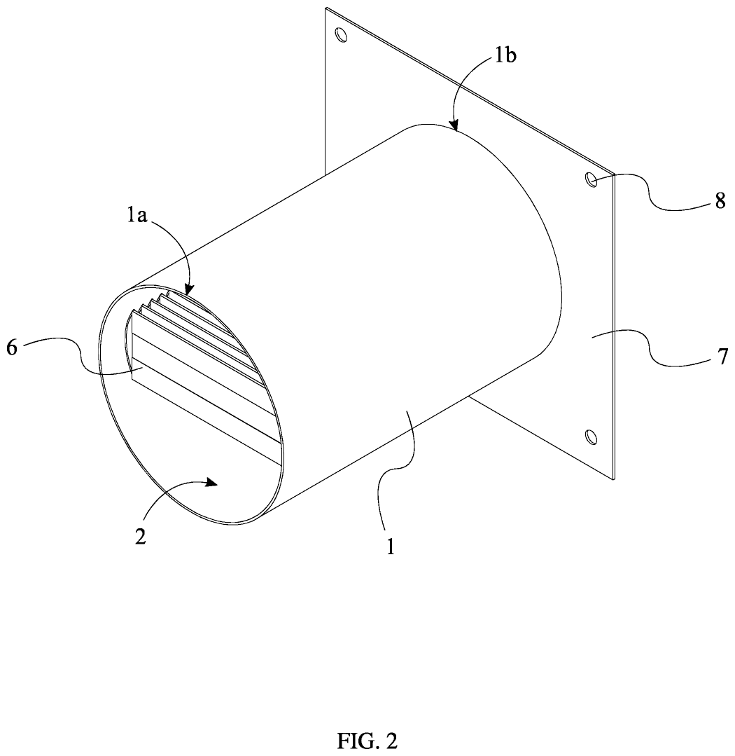

In reference to FIG. 1 through FIG. 6, the present invention is a facemask dispenser. An objective of the present invention is to create an apparatus which may dispense protective equipment such as masks to a user before entering a building or other location. The dispenser allows a user to extract a single use mask without having to touch other surfaces or masks contained in the dispenser. The dispenser is provided a body which houses a plurality of masks and a mounting plate that may allow for the dispenser to be mounted onto a wall. The plurality of masks may be pushed towards a front of the dispenser to an opening where a user can simply grab a mask to wear. Each of the plurality of masks may be contained in a package to further prevent contamination of other masks positioned inside the dispenser. Another method of dispensing masks from the present invention is by having a hook and loop fastener tab that attaches to a mask contained in a package. The user may simply pull a mask out which detaches the mask from the container. A dish may be positioned underneath an opening of the dispenser to catch any masks that could potentially fall. This also prevents the mask from falling out of the dispenser. Furthermore, the dispenser may be positioned in a vertical manner instead of being mounted onto a wall.

The following description is in reference to FIG. 1 through FIG. 6. According to a preferred embodiment, the facemask dispensing device comprises a main body 1, a storage compartment 2, a lid 3, and a dispensing system 4. Preferably, the main body 1 is intended to house facemasks and the associated components needed for the functioning of the present invention. As seen in FIG. 1 through FIG. 6, the main body 1 may be a cylindrical piece oriented in a lateral fashion. Further, the storage compartment 2 traverses normally and centrally through the main body 1. Preferably, the storage compartment 2 houses the facemasks in a protective and non-contaminated environment, and the storage compartment follows the cylindrical shape of the main body 1. However, the main body 1 and the storage compartment 2 may comprise any other shape, size, orientation, components, and arrangement of components that are known to one of ordinary skill in the art, as long as the intents of the present invention are not altered.

It is an aim of the present invention to keep any protective equipment such as facemasks safely and securely within the main body 1. To accomplish this, the lid 3 is mounted onto a first end 1a of the main body 1. In other words, the lid 3 covers the storage compartment 2 and protects the contents of the storage compartment 2 from external elements. Preferably, the lid 3 has dimensions to cover the mouth of the storage compartment 2 and fits perimetrically around the first end 1a. However, the lid 3 may comprise any other size, shape, fastening means, components and arrangement of components that are known to one of ordinary skill in the art, as long as the objectives of the present invention are not altered.

In order for a user to dispense the contents within the storage compartment 2, the dispensing system 4 is mounted onto the main body 1. In the preferred embodiment, the dispensing system 4 is intended to push and move a plurality of face masks 5 towards the first end 1a of the main body 1, so that the user may easily access said plurality of face masks 5 with ease. To that end, the dispensing system 4 is operably coupled to the storage compartment 2, such that the dispensing system 4 dispenses a single facemask 6 at a time from the plurality of facemasks 5 stacked within the storage compartment 2. Preferably, the single facemask 6 is the first facemask from the stacked plurality of facemasks 5, and the single facemask 6 is positioned adjacent the first end 1a of the main body 1.

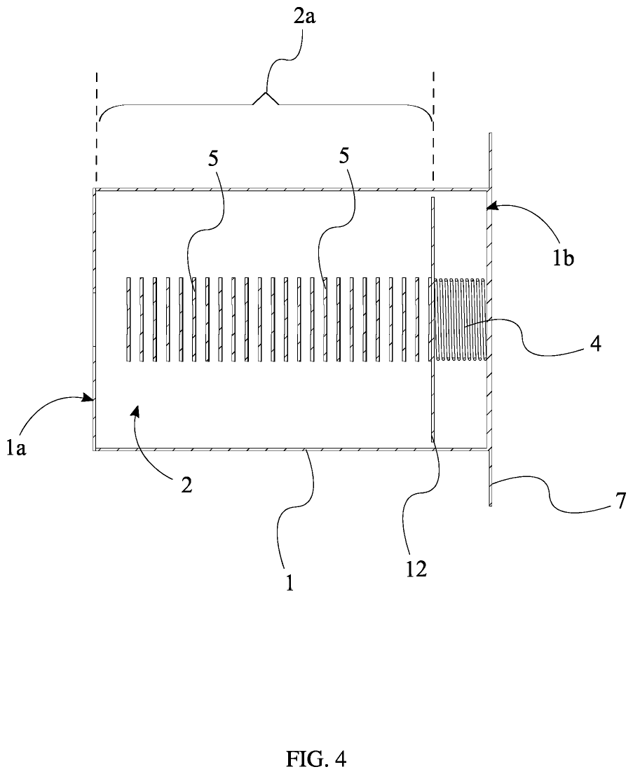

According to the preferred embodiment of the present invention, the dispensing system 4 dispenses a facemask on manual activation. In other words, the present invention may be operated by first introducing the plurality of face masks 5 into the storage compartment 2 via the first end 1a, by opening the lid 3. Each of the plurality of face masks 5 may be contained in a package. This prevents a user from contaminating another member of the plurality of masks 5. The lid 3 of the dispenser may then be closed by rotating the lid about a hinge 3a and may further be secured by a lock or any fastener that secures the lid to the exterior surface of the main body 1. In the preferred embodiment, and as seen in FIG. 4, the dispensing system 4 comprises a loaded spring. However, the dispensing system 4 may comprise any other technology that are known to one of ordinary skill in the art, as long as the intents of the present invention are not altered. For example, in an alternate embodiment of the present invention, the main body 1 of the dispenser may be oriented in a vertical manner. In this embodiment, the dispensing system 4 may comprise a lever on which a user may use an elbow or foot to depress said lever to gain access to one of the plurality of masks 5.

In yet another alternate embodiment, the lid 3 of the present invention may comprise a number pad which allows the user to input a code. If the correct code is dialed into the number pad, the lid 3 will unlock, and the user may then access the plurality of face masks 5.

In yet another alternate embodiment, the dispensing system 4 may comprise a button or other means of a switch. The button activates an aggravator positioned inside the storage compartment 2 to push and move the plurality of face masks 5 towards the first end 1a and allows the user to more easily access said plurality of face masks 5.

Continuing with the preferred embodiment, the present invention comprises a support plate 7 and at least one fastener 8. As seen in FIG. 1 through FIG. 6, the support plate 7 is mounted onto a second end 1b of the main body 1, wherein the second end 1b is positioned opposite to the first end 1a across the main body 1. The support plate 7 is connected to the second end 1b to allow a user to fasten the main body 1 to a wall at an appropriate height to more easily allow access to the dispenser. Further, the storage compartment 2 traverses from the first end 1a towards the second end 1b of the main body 1. In other words, the storage compartment 2 is delineated by the first end 1a and the support plate 7. In the preferred embodiment, the at least one fastener 8 is integrated onto the support plate 7 to enable fastening of the support plate 7 to a convenient wall or supporting surface. Preferably, the at least one fastener 8 comprises screws and nuts. However, any other fastening means and technology, such as adhesives, hook and loop fasteners, clips, magnets etc. may be employed to fasten the support plate 7 to a supporting surface. As seen in FIG. 4, the dispensing device 4 is connected to the support plate 7 and is more specifically positioned within the storage compartment 2.

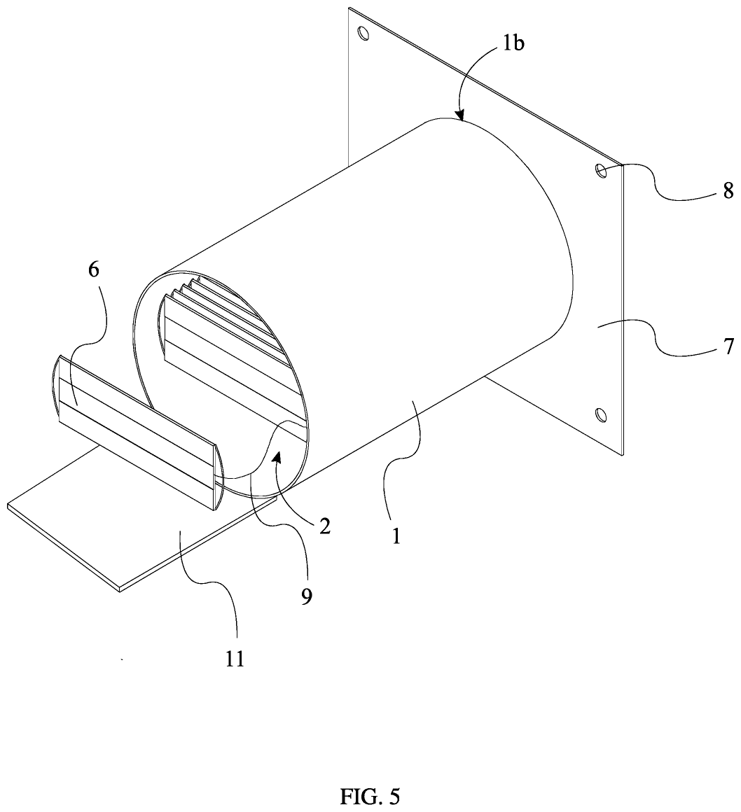

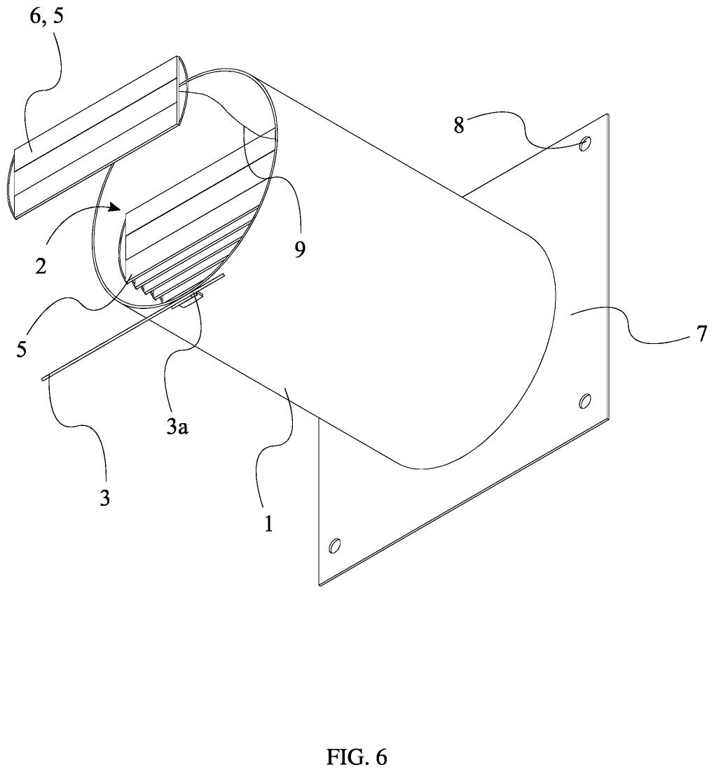

According to the preferred embodiment, the present invention further comprises a stabilizing mechanism 9, wherein the stabilizing mechanism 9 is positioned within the storage compartment 2. Preferably, the stabilizing mechanism 9 is operably coupled to the plurality of facemasks 5, such that the stabilizing mechanism 9 retains the plurality of facemasks 5 in place for dispensing. In other words, the stabilizing mechanism 9 prevents any of the plurality of facemasks 5 from falling out accidentally. For example, an interior surface of the storage compartment 2 may comprise a plurality of attachment points as the stabilizing mechanism 9, such that each member of the plurality of facemasks 5 may be individually packaged are tethered to the main body 1 by a cord. As seen in FIG. 5, and FIG. 6, the stabilizing mechanism 9 is a detachable threaded string in the preferred embodiment. However, the stabilizing mechanism 9 may comprise any other retaining mechanism that are known to one of ordinary skill in the art, as long as the objectives of the present invention are fulfilled.

In an alternate embodiment of the present invention, the stabilizing mechanism 9 as previously described may comprise a hook and loop fastener tab that attached each of the plurality of face masks 5 to the interior surface of the main body 1. More specifically, each of the plurality of face masks 5 may comprise a hook and loop fastener tab which may fasten to one of the plurality of attachment points. This allows the user to more easily detach one of the plurality of masks 5 to be used. This further allows easy replacement of new face masks.

In another alternate embodiment, the stabilizing mechanism 9 may comprise a plurality of shelves or ledges built within the storage compartment 2, such that each of plurality of facemasks 5 may be positioned on a ledge without falling.

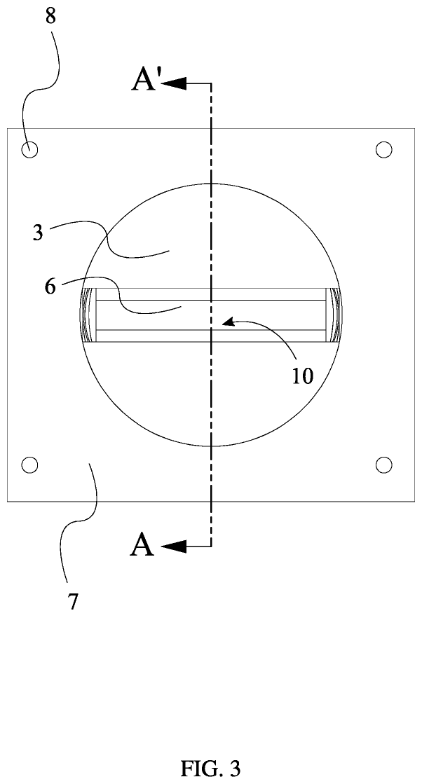

In reference to FIG. 1 and FIG. 3, the present invention comprises a slit 10. Preferably the slit 10 is rectangular in shape and traverses normally through the lid 3. This is so that, the user may pull out one facemask from within the storage compartment 2 through the slit 10. However, the slit 10 may comprise any other shape, size, orientation, components, arrangement of components etc. as long as the intended intentions of the present invention are fulfilled. In the preferred embodiment, the lid 3 is hingedly connected to the first end 1a of the main body 1. This is so that, the user may completely open the lid 3 for taking out a facemask and completely close the lid 3 with ease after taking out the facemask. Accordingly, wherein the lid 3 is in a closed mode, the lid 3 covers the storage compartment 2, thereby protecting the contents of the storage compartment 2 from external elements. Further, wherein the lid 3 is in an open mode, the lid 3 exposes the storage compartment 2, thereby enabling the user to take out the contents within the storage compartment 2 with ease. However, the present invention may employ any other fastening mechanism/technology that are known to one of ordinary skill in the art, to fasten the lid 3 to the main body 1. Examples of such fastening techniques include, but are not limited to threaded screws, snap clips, magnetic clips, hook and loop fasteners etc.

In an alternate embodiment of the present invention, the first end of the main body may comprise a catch plate 11 hingedly attached to the exterior surface of the main body 1, as seen in FIG. 5. The catch plate 11 may catch and hold any of the plurality of face masks 5 that may fall out of the storage compartment 2 of the main body 1.

In reference to FIG. 4, the present invention comprises an inner plate 12. Preferably, the inner plate 12 is mounted within the storage compartment 2 in such a way that the inner plate 12 is terminally connected to the dispensing system 4. The inner plate 12 acts as a panel that receives the plurality of facemasks 5 and being attached to the dispensing system 4, the inner plate 12 moves/pushes the facemasks closer to the user as the dispensing system 4 is being activated. To accomplish this, the inner plate 12 is slidable along a length 2a of the storage compartment 2. As seen in FIG. 4, the plurality of facemasks 5 is positioned adjacent the inner plate 12, opposite to the dispensing system 4. Thus, the present invention is a simple and efficient facemask dispenser that is versatile and user-friendly at the same time.

Although the invention has been explained in relation to its preferred embodiment, it is to be understood that many other possible modifications and variations can be made without departing from the spirit and scope of the invention as hereinafter claimed.

What is claimed is:

1. A facemask dispensing device, the device comprising:
a main body;
a storage compartment;
a dispensing system;
a lid;
a plurality of facemasks;
the storage compartment traversing normally and centrally through the main body;
the dispensing system being mounted onto the main body;
the lid being mounted onto a first end of the main body;
the lid being mounted over the storage compartment;
the plurality of facemasks being stacked within the storage compartment; and
the dispensing system being operably coupled in between the storage compartment and the plurality of masks, wherein the dispensing system dispenses a single facemask at a time from the plurality of facemasks stacked within the storage compartment.

2. The facemask dispensing device of claim 1, wherein the single facemask is the first facemask from the stacked plurality of facemasks, and the single facemask being positioned adjacent the first end of the main body.

3. The facemask dispensing device of claim 1, wherein the dispensing system dispenses a facemask on manual activation.

4. The facemask dispensing device of claim 1, comprising:
a support plate;
at least one fastener;
the support plate being mounted onto a second end of the main body, wherein the second end is positioned opposite to the first end across the main body; and
the at least one fastener being integrated onto the support plate.

5. The facemask dispensing device of claim 4, wherein the storage compartment being delineated by the first end and the support plate.

6. The facemask dispensing device of claim 1, comprising:
a stabilizing mechanism;
the stabilizing mechanism being positioned within the storage cavity; and
the stabilizing mechanism being operably coupled to the plurality of facemasks, such that the stabilizing mechanism retains the plurality of facemasks in an orientation that facilitates dispensing.

7. The facemask dispensing device of claim 6, wherein the stabilizing mechanism being a detachable threaded string.

8. The facemask dispensing device of claim 1, comprising:
a slit; and
the slit traversing normally through the lid.

9. The facemask dispensing device of claim 1, wherein the lid being hingedly connected to the first end of the main body.

10. The facemask dispensing device of claim 1, comprising:
wherein the lid is in a closed mode:
the lid covering the storage cavity.

11. The facemask dispensing device of claim 1, comprising:
wherein the lid is in an open mode:
the lid exposing the storage cavity.

12. The facemask dispensing device of claim 1, comprising:
a catch plate; and
the catch plate being mounted adjacent the first end of the main body.

13. The facemask dispensing device of claim 1, comprising:
an inner plate;
the inner plate being mounted within the storage cavity;
the inner plate being terminally connected to the dispensing system; and
the inner plate being slidable along a length of the storage cavity.

14. The facemask dispensing device of claim 13, wherein the plurality of facemasks being positioned adjacent the inner plate, opposite to the dispensing system.

15. The facemask dispensing device of claim 1, wherein the dispensing system being a loaded spring.

16. A facemask dispensing device, the device comprising:
a main body;
a storage compartment;
a dispensing system;
a lid;
a plurality of facemasks;
a support plate; and
at least one fastener;
the storage compartment traversing normally and centrally through the main body;
the lid being mounted over the storage compartment;
the plurality of facemasks being stacked within the storage compartment;
the dispensing system being mounted onto the main body;
the lid being mounted onto a first end of the main body;
the dispensing system being operably coupled in between the storage compartment and the plurality of masks, wherein the dispensing system dispenses a single facemask at a time from the plurality of facemasks stacked within the storage compartment;
the support plate being mounted onto a second end of the main body, wherein the second end is positioned opposite to the first end across the main body; and
the at least one fastener being integrated onto the support plate.

17. The facemask dispensing device of claim 16, wherein the lid being hingedly connected to the first end of the main body.

18. The facemask dispensing device of claim 16, comprising:
an inner plate;
the inner plate being mounted within the storage compartment;
the inner plate being terminally connected to the dispensing system; and
the inner plate being slidable along a length of the storage compartment.

19. The facemask dispensing device of claim 18, wherein the plurality of facemasks being positioned adjacent the inner plate, opposite to the dispensing system.

20. The facemask dispensing device of claim 16, comprising:
a stabilizing mechanism;
the stabilizing mechanism being positioned within the storage compartment; and
the stabilizing mechanism being operably coupled to the plurality of facemasks, such that the stabilizing mechanism retains the plurality of facemasks in an orientation that facilitates dispensing.
